(19) United States
(12) Patent Application Publication
(10) Pub. No.: US 2024/0319211 A1
(43) Pub. Date: Sep. 26, 2024

(54) CONTENT CONVERSION METHOD OF FREE COMPONENTS BY TRANSFORMING ULTRAFILTRATION TO EQUILIBRIUM DIALYSIS

(71) Applicant: HEFEI NOVA-MS BIOTECH MEDICAL EQUIPMENT CO., LTD, Hefei City, Anhui Province (CN)

(72) Inventors: Jun PENG, Edmonton (CA); Zhenzuo JIANG, Hefei City (CN)

(21) Appl. No.: 18/576,005

(22) PCT Filed: May 8, 2023
(86) PCT No.: PCT/CN2023/092700

§ 371 Date: Jan. 2, 2024

(30) Foreign Application Priority Data
Mar. 22, 2022 (CN) 202210282779.5

(51) Int. Cl. G01N 33/74; B01D 61/14; B01D 61/24; G01N 33/68

(52) U.S. Cl. CPC G01N 33/74; B01D 61/145; B01D 61/243; G01N 33/6848

(57) ABSTRACT

The present invention belongs to the field of free components detection technologies. For sample preparation of free components, although ultrafiltration is efficient and fast, the temperature is not easy to control which result in the measurement results have a large deviation. Conversely, the temperature is easy to control and measurement results are more precise when equilibrium dialysis is used. However, a long time consumed gives rise to equilibrium dialysis cannot meet the requirement for quickly analyzing clinical biological samples. Therefore, this application provides a content conversion method of free components by transforming ultrafiltration to equilibrium dialysis. Free components are separated from samples by ultrafiltration to obtain first concentration. Meanwhile, free components are separated from the samples by equilibrium dialysis to obtain second concentration. A linear equation is established based on the first concentration and the second concentration, which can meet both requirements of high detection accuracy and short time consuming.

### CROSS REFERENCE TO RELATED APPLICATIONS

This application claims priority to and benefits of Chinese Patent Application No. 202210282779.5 filed Mar. 22, 2022, which is incorporated by reference in its entirety.

### TECHNICAL FIELD

The present invention belongs to the field of free components detection technologies, and specifically, relates to a content conversion method of free components by transforming ultrafiltration to equilibrium dialysis.

### BACKGROUND

Free hormones play a variety of biological activity in a human body, which are the dominant part of biological effect of hormones. Currently, the separation methods of free components mainly include equilibrium dialysis and ultrafiltration. Of which, equilibrium dialysis is considered as the gold standard for determination of free components.

For example, the Chinese Patent Application No. CN111398490A entitled “KIT FOR DETECTING FREE TRIIODOTHYRONINE AND FREE THYROXINE BY MASS SPECTROMETRY” includes: reagents used for separating free triiodothyronine and free thyroxine from conjugate triiodothyronine and conjugate thyroxine through equilibrium dialysis, dialysis solution, and reagents used for extracting free triiodothyronine and free thyroxine from dialyzate through solid-phase extraction (SPE). Moreover, the kit contains internal standard solution, equilibrium solution, flushing solution, and elution solution. When using the kit, free triiodothyronine and free thyroxine in human serum are transferred to dialyzate by equilibrium dialysis. Subsequently, the free triiodothyronine and the free thyroxine are extracted from the dialyzate by SPE. Furthermore, above samples are separated and detected by ultra-high performance liquid chromatography and mass spectrometer. Finally, samples concentration are obtained through comparison with calibrators to ensure the accuracy of detection results. And, different concentrations of quality control materials are used to ensure the reliability of the detection results. However, the equilibrium dialysis requires a long time and may change the solution volume. In addition, protein leakage phenomenon may occur due to excessively long dialysis time, which may significantly increase the proportion of free medicine. On the contrary, nonspecific binding existed between the medicine and test apparatuses such as a semi-permeable membrane, which may cause reduce the concentration of the free medicine, and further affect test results. Because of the Donnan effect, charged particles could not pass through semi-permeable membrane which generates uneven charges. In this case, particle concentrations on two sides of semi-permeable membrane are not consistent. In particular, this effect is most pronounced for compounds with high ionization and low protein binding.

Compared with the equilibrium dialysis, ultrafiltration could meet a requirement for quick analysis. For example, the Chinese Patent Application No. CN113390977A entitled “A SIMULTANEOUSLY DETERMINATION METHOD OF FREE THYROID HORMONES (T3, rT3, AND T4) AND CORTISOL IN SALIVA” adopts ultrafiltration. A proper filter membrane within a molecular weight cut-off limit range is selected to remove proteins, conjugate thyroid hormones and cortisol in the saliva. Subsequently, a proper proportion of anti-adsorption reagents such as methanol are added to solve the severe adsorption problem which exists in the detection process of free T3, rT3, T4 and cortisol in the saliva. The free thyroid hormones (T3, rT3 and T4) and cortisol in the saliva could be simultaneously detected precisely by using a high-performance liquid chromatography tandem mass spectrometry. The method has advantages of high throughput, simple operation, high efficiency, low cost, high accuracy and high sensitivity. However, temperature is not easily controlled during the ultrafiltration process. Meanwhile, the concentration of free hormones could be changed with temperature during ultrafiltration or equilibrium dialysis. Since the human body temperature is 37° C., temperature of ultrafiltration or equilibrium dialysis at 37° C. can better reflect the content and physiological activity of the human free hormones. However, the temperature of existing centrifuge cannot be quickly stabilized or cannot be reached 37° C.

As described above, a long time is consumed when the equilibrium dialysis is employed. For example, free hormonal components in blood usually need to be incubated in a thermostat at 37° C. for at least 22 h, which cannot meet the requirements for quickly analyzing results of clinical biological samples. In addition, because of a small volume of dialysate, low-content free components are difficult to be detected by conventional equilibrium dialysis even though these free components are enriched. On the contrary, larger volume can be obtained by ultrafiltration. And, the concentration of free components in the ultrafiltrate is not affected by volume migration. Therefore, a detection limit can be significantly improved after enrichment and concentration, while temperature cannot be controlled easily in the ultrafiltration process.

### SUMMARY

### 1. To-be-Resolved Problem

For sample preparation of free components, although ultrafiltration is efficient and fast, the temperature is not easy to control which result in the measurement results have a large deviation. On the contrary, measurement results are more precise when equilibrium dialysis is used. However, a long time consumed gives rise to equilibrium dialysis cannot meet the requirement for quickly analyzing clinical biological samples. Therefore, this application provides a content conversion method of free components by transforming ultrafiltration to equilibrium dialysis, which can meet both requirements of high detection accuracy and short time consuming.

### 2. Technical Solutions

To achieve the foregoing objectives, the technical solutions provided as follows:

According to the present invention, a content conversion method of free components by transforming ultrafiltration to equilibrium dialysis, including the following steps:
a step of separating the free components from samples by ultrafiltration and measuring concentrations of free components, to obtain first concentration;
a step of separating the free components from samples by equilibrium dialysis and measuring concentrations of free components, to obtain second concentration; and
a step of establishing a linear equation based on the first concentration and the second concentration.

Preferably, measurement results of equilibrium dialysis at a specific temperature (4° C. to 37° C.) are used as a horizontal coordinate (x), and measurement results of ultrafiltration at a specific temperature (4° C. to 37° C.) are used as a vertical coordinate (y), and linear regression is performed by using a least square method, to obtain a linear equation of measurement results of ultrafiltration at the specific temperature and results of equilibrium dialysis at the specific temperature.

Further, there are a plurality of samples, and the quantity of samples is sufficient for a correlation coefficient of the established linear equation to be greater than 0.90.

Preferably, at least two samples need to be used to establish the linear equation. Preferably, 20 samples are selected to establish the linear equation. More preferably, more than 50 samples are selected to establish the linear equation. Most preferably, more than 100 samples are selected to establish the linear equation.

Preferably, the correlation coefficient of linear equation is greater than 0.95.

Further, high-performance liquid chromatography tandem mass spectrometry is used for the step of measuring concentrations.

In another implementation, gas chromatography tandem mass spectrometry is used for the step of measuring concentrations.

Further, the first concentration and the second concentration are measured at different temperatures.

Further, the first concentration is measured at 4° C. to 37° C.; and the second concentration data is measured at 4° C. to 37° C.

Preferably, the first concentration is measured at 25° C.; and the second concentration is measured at 37° C.

Further, the sample is blood, saliva, or urine.

Further, the sample is serum or plasma.

Further, the free components are free hormones.

Further, the free hormones are free testosterone, free triiodothyronine, or free thyroxine.

Further, when the first concentration is measured at 25° C. and the second concentration is measured at 37° C., the linear equation is y=0.8719x−0.2116 when the free hormone is free testosterone; the linear equation is y=0.7745x+0.066 when the free hormone is free triiodothyronine; the linear equation is y=0.6205x+0.3418 when the free hormone is free thyroxine; and y represents the first concentration, and x represents the second concentration.

### 3. Beneficial Effects

The technical solutions provided in the present invention have the following beneficial effects compared with the known technologies:

In the content conversion method of free components by transforming ultrafiltration to equilibrium dialysis according to the present invention, the free components are separated from the sample by ultrafiltration to obtain the first concentration, the free components are separated from the sample by equilibrium dialysis to obtain the second concentration, and the linear equation is established based on the first concentration and the second concentration, to convert measured data of ultrafiltration to the measured data of equilibrium dialysis, which can meet both requirements of high detection accuracy and short time consuming. The concentrations of free analytes in samples can be measured through ultrafiltration at ambient temperature condition. The method is easy to operate and is not affected by sample dilution and volume migration.

In the content conversion method of free components by transforming ultrafiltration to equilibrium dialysis according to the present invention, the concentrations of the free hormones in the serum can be rapidly analyzed by ultrafiltration, and obtained volume of ultrafiltrate is also much more than dialyzate, thereby improving the detection limit of free hormones.

In the content conversion method of free components by transforming ultrafiltration to equilibrium dialysis according to the present invention, theoretical concentrations of free hormones at 37° C. can be converted by ultrafiltration at 25° C., this temperature is easily controlled, the results are not affected by volume migration, and the detection results are more reliable.

In the content conversion method of free components by transforming ultrafiltration to equilibrium dialysis according to the present invention, free hormones (such as testosterone, thyroxine and triiodothyronine) are measured by ultrafiltration coupled to LC-MS/MS. In the conventional equilibrium dialysis, because of a small volume of dialysate, low-content free components are difficult to be detected, even though these free components are enriched. On the contrary, larger volume can be obtained by ultrafiltration. And, the concentration of free components in the ultrafiltrate is not affected by volume migration. Therefore, a detection limit can be significantly improved after enrichment and concentration.

### BRIEF DESCRIPTION OF DRAWINGS

FIG. 1 shows the linear result of free testosterone at conditions of 25° C. ultrafiltration and 37° C. equilibrium dialysis;

FIG. 2 shows the linear result of free T3 at conditions of 25° C. ultrafiltration and 37° C. equilibrium dialysis; and

FIG. 3 shows the linear result of free T4 at conditions of 25° C. ultrafiltration and 37° C. equilibrium dialysis.

### DESCRIPTION OF EMBODIMENTS

To further understand the content of the present invention, the present invention is described in detail with reference to the embodiments.

### 1. Experimental Materials Used in the Embodiments are as Follows:

### 1.1. Reference Substances and Reagents

Methanol (HPLC grade, Merck), acetonitrile (HPLC grade, Merck), formic acid (HPLC grade, Aladdin), ammonium acetate (HPLC grade, Aladdin), sodium chloride (AR grade, SCR), potassium dihydrogen phosphate (AR grade, SCR), magnesium sulfate heptahydrate (AR grade, SCR), 4-hydroxyethyl piperazine ethanesulfonic acid (AR grade, Ourchem), urea (AR grade, SCR), calcium chloride dihydrate (AR grade, SCR), and sodium hydroxide (AR grade, SCR).

### 1.2. Main Instruments, Equipments, and Consumables Used in the Embodiments are as Follows:

LC-MS/MS: Waters UPLC-I Class ultra-high performance liquid chromatograph and AB Sciex 5500 mass spectrometer (AB Sciex);
Chromatographic column: ACQUITY UPLC BEH C18 (2.1×50 mm, 1.7 μm) (Waters); and
vortex mixer (SI Vortex Genie 2, United States), low-speed freezing centrifuge (USTC Zonkia), ultrafiltration device (30 kDa), 96-well disposable equilibrium dialysis plate (30 kDa), analysis balance (0.01 mg, Mettler Toledo, Switzerland), and electrothermal standing-temperature incubator (ShangCheng Instrument).

### 2. Solutions and Reagents Preparation

### 2.1. Preparation of a 4-Hydroxyethyl Piperazine Ethanesulfonic Acid Solution

5.26 g sodium chloride, 224 mg potassium dihydrogen phosphate, 275 mg magnesium sulfate heptahydrate, 12.57 g 4-hydroxyethyl piperazine ethanesulfonic acid, 300 mg urea, 275 mg calcium dihydrate chloride, and 900 mg sodium hydroxide are precisely weighted and placed in a beaker. 1 L pure water is added and stirring well. Subsequently, the 4-hydroxyethyl piperazine ethanesulfonic acid buffer solution is obtained after still standing.

### 2.2. Activating Solution Preparation

400 mg sodium hydroxide is weighted and dissolved in 100 mL water to prepare 0.1 M NaOH activating solution.

### 3. Apparatus Preparation

### 3.1. Activation of Ultrafiltration Tube

100 μL 0.1 M NaOH activating solution is placed in an ultrafiltration tube and centrifuged at 25° C. and 2000 g for 10 min. Subsequently, 100 μL pure water is added and centrifuged at the same conditions.

### 3.2. Activation of Solid-Phase Extraction

Solid-phase extraction is activated by using 1 mL methanol and 1 mL pure water, respectively.

### Embodiment 1

In a content conversion method of free components by transforming ultrafiltration to equilibrium dialysis according to this embodiment, a sample is free testosterone.

In the method in this embodiment, ultrafiltration and equilibrium dialysis are performed to separate free testosterone at 20° C., 25° C., 30° C., and 37° C., respectively. In practice, other temperatures can also be used. Subsequently, high-performance liquid chromatography tandem mass spectrometry is used for detection, and results are shown in Table 1. Where

A specific solution used for ultrafiltration is as follows:

Ultrafiltration: 900 μL 4-hydroxyethyl piperazine ethanesulfonic acid solution is added to 300 μL serum, and the solution is mixed well by pipettes. Subsequently, the obtained mixture is transferred to the activated ultrafiltration tube and centrifuged at 20° C., 25° C., 30° C., and 37° C., and 2000 g for 1 h, respectively.

Solid-phase extraction: 20 μL internal standard solution is added to 600 μL ultrafiltrate, and vortexed well. The obtained mixture is added to an activated HLB solid-phase extraction: conditioning is performed twice by 300 μL 10% methanol aqueous solution, elution is performed twice by 300 μL 90% methanol aqueous solution, eluent is merged and blown to dry by nitrogen, and finally, the obtained product is redissolved by 100 μL 50% methanol aqueous solution.

A specific solution used for equilibrium dialysis is as follows:

Equilibrium dialysis: 200 μL 4-hydroxyethyl piperazine ethanesulfonic acid solution is placed on the buffer side of the equilibrium dialysis plate, 200 μL serum is placed on the sample side. Subsequently, the equilibrium dialysis plate is dialyzed at 20° C., 25° C., 30° C., and 37° C. for 22 h, respectively.

Solid-phase extraction: 20 μL internal standard solution is added to 200 μL dialysate, and vortexed well. The obtained mixture is added to an activated HLB solid-phase extraction: conditioning is performed twice by 300 μL 10% methanol aqueous solution, elution is performed twice by using 300 μL 90% methanol aqueous solution, eluent is merged and blown to dry by nitrogen, and finally, the obtained product is redissolved by 100 μL 50% methanol aqueous solution.

After the free testosterone is separated by ultrafiltration and equilibrium dialysis, the free testosterone is detected by the following high-performance liquid chromatography tandem mass spectrometry parameters:

### Chromatographic Conditions:

Chromatographic column: Acquity UPLC BEH C18 (2.1×50 mm, 1.7 μm); column oven temperature: 40° C.; mobile phases: 0.1% formic acid 2 mM ammonium acetate aqueous solution (A)˜methanol (B); and gradient elution:

| Time, | Flow rate, | Mobile phase A, | Mobile phase B, |
|---|---|---|---|
| min | mL/min | % | % |
| Initial | 0.600 | 95 | 5 |
| 1.20 | 0.600 | 5 | 95 |
| 1.40 | 0.600 | 5 | 95 |
| 1.41 | 0.600 | 95 | 5 |
| 1.60 | 0.600 | 95 | 5 |

### Mass Spectrometry Conditions:

ion source: electrospray ion source in positive ion mode; spray voltage: 5500 V; ion source temperature: 550° C.; GS1: 50 psi; GS2: 50 psi; curtain gas: 30 psi; and multiple reaction monitoring:

| | Parent ion, | Daughter ion, | DP, | CE, |
|---|---|---|---|---|
| Analyte | m/z | m/z | V | eV |
| Testosterone* | 289.3 | 109.1 | 40 | 15 |
| Testosterone | 289.3 | 97.1 | 40 | 15 |
| Testosterone -d3 | 292.2 | 109.1 | 40 | 15 |

The measurement results of the free testosterone at the above-mentioned LC-MS/MS conditions are shown in Table 1:

TABLE 1
The measurement results of the free testosterone for
the same samples obtained by ultrafiltration (UF) and
equilibrium dialysis (ED) at different temperatures

| | Temp. | | | | | | | |
|---|---|---|---|---|---|---|---|---|
| | 20° C. | | 25° C. | | 30° C. | | 37° C. | |
| Sample | Concentration: pg/mL | | | | | | | |
| No. | UF | ED | UF | ED | UF | ED | UF | ED |
| 1 | 4.26 | 4.05 | 4.37 | 4.75 | 4.69 | 5.04 | 5.12 | 5.40 |
| 2 | 4.29 | 4.17 | 4.64 | 4.61 | 4.96 | 5.01 | 5.28 | 4.95 |
| 3 | 4.01 | 3.94 | 4.51 | 4.48 | 4.83 | 4.95 | 5.30 | 5.23 |
| 4 | 4.38 | 4.21 | 4.45 | 4.56 | 4.79 | 5.02 | 5.04 | 5.42 |
| 5 | 3.98 | 3.85 | 4.39 | 4.41 | 4.58 | 4.85 | 5.16 | 5.12 |
| 6 | 3.92 | 4.18 | 4.64 | 4.62 | 4.96 | 4.87 | 5.18 | 5.32 |
| 7 | 4.15 | 4.01 | 4.51 | 4.57 | 4.85 | 4.91 | 5.24 | 5.22 |
| 8 | 4.12 | 4.16 | 4.48 | 4.42 | 4.91 | 4.98 | 5.16 | 5.27 |
| 9 | 3.97 | 4.05 | 4.43 | 4.50 | 4.74 | 4.84 | 5.03 | 5.13 |
| 10 | 4.07 | 4.22 | 4.61 | 4.54 | 4.77 | 4.82 | 5.07 | 5.07 |
| Average | 4.12 | 4.08 | 4.50 | 4.55 | 4.81 | 4.93 | 5.16 | 5.21 |

| | | | | |
|---|---|---|---|---|
| UF/ED | 101.0% | 98.90% | 97.56% | 99.04% |

Table 1 shows that the measurement results of the free testosterone obtained by ultrafiltration and equilibrium dialysis are consistent at a plurality of different temperature conditions. In other words, the ultrafiltration and the equilibrium dialysis have consistent results of free testosterone at the same temperature. Therefore, the equilibrium dialysis at 37° C. can be used as reference to establish a conversion formula between the ultrafiltration at different temperatures (for example, 20° C., 25° C., and 30° C.) and the equilibrium dialysis at 37° C. Finally, the present patent achieves the content conversion of free testosterone by transforming ultrafiltration at different temperatures (for example, 20° C., 25° C., and 30° C.) to the concentrations obtained by equilibrium dialysis at 37° C. or ultrafiltration at 37° C.

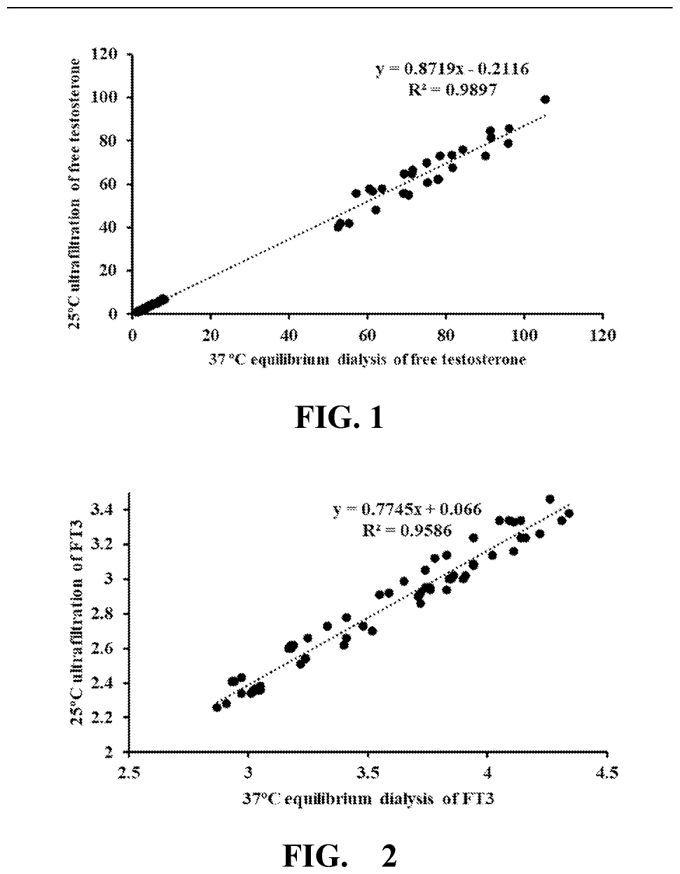

For ease of further understanding, in this embodiment, comparison the concentrations of free testosterone between ultrafiltration at 25° C. and equilibrium dialysis at 37° C. is merely used as an example (similar comparison may be performed at other temperatures). In detail, concentrations of free testosterone in 60 samples (30 females and 30 males) are measured by ultrafiltration at 25° C. and equilibrium dialysis at 37° C., respectively. Measurement results are shown in Table 2, and the obtained calculation formula is shown in FIG. 1.

TABLE 2
Measurement results of free testosterone in 60
different serum specimens under 25° C. ultrafiltration
(UF) and 37° C. equilibrium dialysis (ED)

| No. | 1st conc. (UF) | 2nd conc. (ED) |
|---|---|---|
| 1 | 1.21 | 1.33 |
| 2 | 1.52 | 1.92 |
| 3 | 5.00 | 5.35 |
| 4 | 6.48 | 7.79 |
| 5 | 1.60 | 2.05 |
| 6 | 3.88 | 4.17 |
| 7 | 3.40 | 4.06 |
| 8 | 1.84 | 1.93 |
| 9 | 2.43 | 3.22 |
| 10 | 3.23 | 3.59 |
| 11 | 5.10 | 6.36 |
| 12 | 1.65 | 2.19 |
| 13 | 3.90 | 4.25 |
| 14 | 2.83 | 3.05 |
| 15 | 5.44 | 6.73 |
| 16 | 6.17 | 6.83 |
| 17 | 2.11 | 2.62 |
| 18 | 1.94 | 2.40 |
| 19 | 1.05 | 1.31 |
| 20 | 1.44 | 1.53 |
| 21 | 1.05 | 1.31 |
| 22 | 1.44 | 1.53 |
| 23 | 6.80 | 8.25 |
| 24 | 4.52 | 5.38 |
| 25 | 1.66 | 1.79 |
| 26 | 4.17 | 4.63 |
| 27 | 6.42 | 7.41 |
| 28 | 1.33 | 1.43 |
| 29 | 1.97 | 2.08 |
| 30 | 3.89 | 4.82 |
| 31 | 7.16 | 7.77 |
| 32 | 1.20 | 1.56 |
| 33 | 78.76 | 95.83 |
| 34 | 73.23 | 78.52 |
| 35 | 62.35 | 78.15 |
| 36 | 40.06 | 52.51 |
| 37 | 64.79 | 69.37 |
| 38 | 81.34 | 91.57 |
| 39 | 57.89 | 63.64 |
| 40 | 54.80 | 70.49 |
| 41 | 76.10 | 84.31 |
| 42 | 60.66 | 75.24 |
| 43 | 56.61 | 61.24 |
| 44 | 66.50 | 71.47 |
| 45 | 84.61 | 91.37 |
| 46 | 55.67 | 69.46 |
| 47 | 73.21 | 90.17 |
| 48 | 41.92 | 55.38 |
| 49 | 55.83 | 57.02 |
| 50 | 62.18 | 77.82 |
| 51 | 57.89 | 60.57 |
| 52 | 67.79 | 81.72 |
| 53 | 85.59 | 96.20 |
| 54 | 47.93 | 62.16 |
| 55 | 64.80 | 71.35 |
| 56 | 69.87 | 75.17 |
| 57 | 73.44 | 81.44 |
| 58 | 55.69 | 69.19 |
| 59 | 99.22 | 105.37 |
| 60 | 42.07 | 53.15 |

The results indicate that the concentrations of the free testosterone in 60 serum specimens are acquired by ultrafiltration at 25° C. for 1 h and equilibrium dialysis at 37° C. for 22 h, respectively, to obtain ultrafiltrate (first concentrations, 1st conc.) and equilibrium dialyzate (second concentrations, 2nd conc.), and finally, a related linear equation is obtained. The linear equation of the free testosterone is finally calculated based on the results of 25° C. ultrafiltration and the results of 37° C. equilibrium dialysis, which is y=0.8719x−0.2116, where R²=0.9897, and n=60. The coefficient of determination R² is greater than 0.95, which indicates good correlation between the results of 25° C. ultrafiltration and the results of 37° C. equilibrium dialysis. Then, the concentration of free testosterone at 37° C. (ultrafiltration or equilibrium dialysis) may be calculated based on the result of 25° C. ultrafiltration, and calculation formulas are as follows:

the first concentration obtained at 25° C. ultrafiltration=the second concentration obtained at 37° C.

$$equilibrium dialysis \times 0.8719 - 0.2116 ; and$$

$$a sample concentration = ( the first concentration + 0.2116 ) / 0.871 9 .$$

### Embodiment 2

In a method in this embodiment, ultrafiltration and equilibrium dialysis are performed to separate free triiodothyronine (FT3) at 20° C., 25° C., 30° C., and 37° C., respectively. In practice, another temperature value may be used. Then, high-performance liquid chromatography tandem mass spectrometry is used for detection, and results are shown in Table 3. Where

A specific solution used for ultrafiltration is as follows:

Ultrafiltration: 100 μL 4-hydroxyethyl piperazine ethanesulfonic acid solution is added to a 100 μL serum, and the solution is mixed well by pipettes. Subsequently, the obtained mixture is transferred to the activated ultrafiltration tube and centrifuged at 20° C., 25° C., 30° C., and 37° C., and 2000 g for 1 h, respectively.

Internal standard precipitation: 100 μL internal standard solution is added to 100 μL ultrafiltrate, and vortexed well for 3 min. Then, centrifugation is performed at 4° C. and 12000 rpm for 10 min.

A specific solution used for equilibrium dialysis is as follows:

Equilibrium dialysis: 200 μL 4-hydroxyethyl piperazine ethanesulfonic acid solution is placed on the buffer side of the equilibrium plate, 200 μL serum is placed on the sample side. Subsequently, the equilibrium dialysis plate is dialyzed at 20° C., 25° C., 30° C., and 37° C. for 22 h, respectively.

Internal standard precipitation: 100 μL internal standard solution is added to 100 μL dialysate and vortexed well for 3 min. Then, centrifugation is performed at 4° C. and 12000 rpm for 10 min.

After the free T3 is separated by using ultrafiltration and equilibrium dialysis, the free T3 is detected by the following high-performance liquid chromatography tandem mass spectrometry parameters:

### Chromatographic Conditions:

chromatographic column: Acquity UPLC BEH C18 (2.1×50 mm, 1.7 μm); column oven temperature: 30° C.; mobile phases: 0.1% formic acid aqueous solution (A)˜0.1% formic acid methanol (B); and gradient elution:

| Time, | Flow rate, | Mobile phase A, | Mobile phase B, |
|---|---|---|---|
| min | mL/min | % | % |
| 0 | 0.300 | 80 | 20 |
| 1 | 0.300 | 80 | 20 |
| 3 | 0.300 | 0 | 100 |
| 3.1 | 0.300 | 80 | 20 |
| 4 | 0.300 | 80 | 20 |

### Mass Spectrometry Conditions:

ion source: electrospray ion source in positive ion mode; spray voltage: 5500 V; ion source temperature: 550° C.; GS1: 50 psi; GS2: 50 psi; curtain gas: 30 psi; and multiple reaction monitoring:

| Analyte | Parent ion, m/z | Daughter ion, m/z | DP, V | CE, eV |
|---|---|---|---|---|
| FT3* | 651.7 | 605.7 | 100 | 30 |
| FT3 | 651.7 | 478.7 | 100 | 40 |
| ¹³C₆-FT3 | 657.7 | 611.7 | 100 | 30 |

The measurement results of the FT3 at the above-mentioned LC-MS/MS conditions are shown in Table 3:

TABLE 3
The measurement results of the FT3 for the same
samples obtained by ultrafiltration (UF) and equilibrium
dialysis (ED) at different temperatures

| | Temp. | | | | | | | |
|---|---|---|---|---|---|---|---|---|
| | 20° C. | | 25° C. | | 30° C. | | 37° C. | |
| Sample | Concentration: pg/mL | | | | | | | |
| No. | UF | ED | UF | ED | UF | ED | UF | ED |
| 1 | 2.54 | 2.45 | 2.52 | 2.65 | 2.68 | 2.72 | 3.05 | 3.13 |
| 2 | 2.25 | 2.62 | 2.62 | 2.75 | 2.75 | 2.65 | 3.24 | 3.22 |
| 3 | 2.36 | 2.25 | 2.45 | 2.52 | 2.82 | 2.85 | 3.13 | 3.04 |
| 4 | 2.41 | 2.35 | 2.68 | 2.61 | 2.66 | 2.88 | 3.48 | 3.32 |
| 5 | 2.32 | 2.48 | 2.58 | 2.48 | 2.78 | 2.68 | 3.20 | 3.15 |
| 6 | 2.28 | 2.36 | 2.46 | 2.54 | 2.58 | 2.76 | 3.18 | 3.31 |
| 7 | 2.17 | 2.27 | 2.57 | 2.68 | 2.84 | 2.78 | 3.27 | 3.34 |
| 8 | 2.52 | 2.46 | 2.65 | 2.70 | 2.75 | 2.85 | 3.08 | 3.28 |
| 9 | 2.36 | 2.42 | 2.71 | 2.63 | 2.72 | 2.78 | 3.17 | 3.12 |
| 10 | 2.23 | 2.31 | 2.53 | 2.57 | 2.67 | 2.71 | 3.05 | 3.31 |
| Average | 2.34 | 2.40 | 2.58 | 2.61 | 2.73 | 2.77 | 3.19 | 3.22 |

| | | | | |
|---|---|---|---|---|
| UF/ED | 97.50% | 98.85% | 98.55% | 99.07% |

Table 3 shows that the measurement results of the FT3 obtained by ultrafiltration and equilibrium dialysis are consistent at a plurality of different temperature conditions. In other words, the ultrafiltration and the equilibrium dialysis have consistent results of FT3 at the same temperature. Therefore, the equilibrium dialysis at 37° C. can be used as reference to establish a conversion formula between the ultrafiltration at different temperatures (for example, 20° C., 25° C., and 30° C.) and the equilibrium dialysis at 37° C., Finally, the present patent achieves the content conversion of FT3 by transforming ultrafiltration at different temperatures (for example, 20° C., 25° C., and 30° C.) to the concentration obtained by equilibrium dialysis at 37° C. or ultrafiltration at 37° C.

For ease of further understanding, in this embodiment, comparison the concentrations of FT3 between ultrafiltration at 25° C. and equilibrium dialysis at 37° C. is merely used as an example (similar comparison may be performed at other temperatures). In detail, concentrations of FT3 in 60 samples are measured by ultrafiltration at 25° C. and equilibrium dialysis at 37° C., respectively. Measurement results are shown in Table 4, and the obtained calculation formula is shown in FIG. 2.

TABLE 4
Measurement results of the FT3 in 60 different
serum specimens under 25° C. ultrafiltration
(UF) and 37° C. equilibrium dialysis (ED)

| No. | 1st conc. (UF) | 2nd conc. (ED) |
|---|---|---|
| 1 | 2.36 | 3.02 |
| 2 | 3.09 | 3.94 |
| 3 | 2.43 | 2.97 |
| 4 | 3.14 | 3.83 |
| 5 | 3.24 | 3.94 |
| 6 | 3.34 | 4.05 |
| 7 | 2.37 | 3.03 |
| 8 | 2.36 | 3.05 |
| 9 | 3.02 | 3.86 |
| 10 | 3.38 | 4.34 |
| 11 | 3.02 | 3.91 |
| 12 | 2.41 | 2.93 |
| 13 | 3.05 | 3.74 |
| 14 | 2.60 | 3.18 |
| 15 | 2.34 | 3.01 |
| 16 | 2.94 | 3.76 |
| 17 | 2.95 | 3.74 |
| 18 | 3.26 | 4.22 |
| 19 | 3.34 | 4.14 |
| 20 | 3.46 | 4.26 |
| 21 | 2.60 | 3.17 |
| 22 | 2.70 | 3.52 |
| 23 | 2.92 | 3.59 |
| 24 | 3.14 | 4.02 |
| 25 | 2.73 | 3.33 |
| 26 | 2.51 | 3.22 |
| 27 | 2.90 | 3.71 |
| 28 | 2.36 | 3.04 |
| 29 | 3.00 | 3.84 |
| 30 | 3.16 | 4.11 |
| 31 | 3.24 | 4.16 |
| 32 | 3.34 | 4.09 |
| 33 | 2.66 | 3.25 |
| 34 | 2.54 | 3.24 |
| 35 | 2.94 | 3.83 |
| 36 | 2.41 | 2.94 |
| 37 | 2.78 | 3.41 |
| 38 | 2.91 | 3.55 |
| 39 | 3.08 | 3.94 |
| 40 | 2.62 | 3.19 |
| 41 | 2.35 | 3.02 |
| 42 | 3.24 | 4.14 |
| 43 | 3.33 | 4.11 |
| 44 | 3.00 | 3.85 |
| 45 | 2.73 | 3.48 |
| 46 | 2.54 | 3.24 |
| 47 | 2.95 | 3.76 |
| 48 | 3.34 | 4.31 |
| 49 | 3.00 | 3.90 |
| 50 | 2.62 | 3.18 |
| 51 | 2.99 | 3.65 |
| 52 | 3.12 | 3.78 |
| 53 | 2.92 | 3.72 |
| 54 | 2.62 | 3.40 |
| 55 | 2.86 | 3.72 |
| 56 | 2.66 | 3.41 |
| 57 | 2.34 | 2.97 |
| 58 | 2.28 | 2.91 |
| 59 | 2.38 | 3.05 |
| 60 | 2.26 | 2.87 |

The results indicate that the concentrations of FT4 in 60 serum specimens are acquired by ultrafiltration at 25° C. for 1 h and equilibrium dialysis at 37° C. for 22 h, respectively, to obtain ultrafiltrate (first concentrations, 1st conc.) and equilibrium dialyzate (second concentrations, 2nd conc.), and finally, a related linear equation is obtained. The linear equation of the FT3 is finally calculated based on the results of 25° C. ultrafiltration and the results of 37° C. equilibrium dialysis, which is y=0.7745x+0.066, where R²=0.9586, and n=60. The coefficient of determination R² is greater than 0.95, which indicates good correlation between the results of 25° C. ultrafiltration and the results of 37° C. equilibrium dialysis. Then, the concentration of FT3 37° C. (ultrafiltration or equilibrium dialysis) may be calculated based on the result of 25° C. ultrafiltration, and calculation formulas are as follows:

$$the first concentration ontained at 25 ° C . ultrafiltration = the second concentration obtained at 37 ° C .$$

$$equilibrium dialysis \times 0.7745 + 0.066 ; and$$

$$a sample concentration = ( the first concentration - 0.066 ) / 0.774 5 .$$

### Embodiment 3

In the method in this embodiment, ultrafiltration and equilibrium dialysis are performed to separate free thyroxine (FT4) at 20° C., 25° C., 30° C., and 37° C., respectively. In practice, other temperatures can also be used. Subsequently, high-performance liquid chromatography tandem mass spectrometry is used for detection, and results are shown in Table 5. Where

A specific solution used for ultrafiltration is as follows:

Ultrafiltration: 100 μL 4-hydroxyethyl piperazine ethanesulfonic acid solution is added to 100 μL serum, and the solution is mixed well by pipettes. Subsequently, the obtained mixture is transferred to the activated ultrafiltration tube and centrifuged at 20° C., 25° C., 30° C., and 37° C., and 2000 g for 1 h, respectively.

Internal standard precipitation: 100 μL internal standard solution is added to 100 μL ultrafiltrate, and vortexed well for 3 min. Then, centrifugation is performed at 4° C. and 12000 rpm for 10 min.

A specific solution used for equilibrium dialysis is as follows:

Equilibrium dialysis: 200 μL 4-hydroxyethyl piperazine ethanesulfonic acid solution is placed on the buffer side of the equilibrium dialysis plate, 200 μL serum is placed on the sample side. Subsequently, the equilibrium dialysis plate is dialyzed at 20° C., 25° C., 30° C., and 37° C. for 22 h, respectively.

Internal standard precipitation: 100 μL internal standard solution is added to 100 μL ultrafiltrate, and vortexed well for 3 min. Then, centrifugation is performed at 4° C. and 12000 rpm for 10 min.

After the FT4 is separated by ultrafiltration and equilibrium dialysis, the FT4 is detected by the following high-performance liquid chromatography tandem mass spectrometry parameters:

### Chromatographic Conditions:

chromatographic column: Acquity UPLC BEH C18 (2.1×50 mm, 1.7 μm); column oven temperature: 30° C.; mobile phases: 0.1% formic acid aqueous solution (A)˜0.1% formic acid methanol (B); and gradient elution:

| Time, | Flow rate, | Mobile phase A, | Mobile phase B, |
|---|---|---|---|
| min | mL/min | % | % |
| 0 | 0.300 | 80 | 20 |
| 1 | 0.300 | 80 | 20 |
| 3 | 0.300 | 0 | 100 |
| 3.1 | 0.300 | 80 | 20 |
| 4 | 0.300 | 80 | 20 |

### Mass Spectrometry Conditions:

ion source: electrospray ion source in positive ion mode; spray voltage: 5500 V; ion source temperature: 550° C.; GS1: 50 psi; GS2: 50 psi; curtain gas: 30 psi; and multiple reaction monitoring:

| Analyte | Parent ion, m/z | Daughter ion, m/z | DP, V | CE, eV |
|---|---|---|---|---|
| FT4* | 777.7 | 731.7 | 110 | 35 |
| FT4 | 777.7 | 604.9 | 110 | 45 |
| ¹³C₆-FT4 | 783.7 | 737.7 | 110 | 35 |

The measurement results of the FT4 at the above-mentioned LC-MS/MS conditions are shown in Table 5:

TABLE 5
The measurement results of the FT4 for the same
samples obtained by ultrafiltration (UF) and equilibrium
dialysis (ED) at different temperatures

| | Temp. | | | | | | | |
|---|---|---|---|---|---|---|---|---|
| | 20° C. | | 25° C. | | 30° C. | | 37° C. | |
| Sample | Concentration: pg/mL | | | | | | | |
| No. | UF | ED | UF | ED | UF | ED | UF | ED |
| 1 | 7.21 | 7.05 | 7.92 | 8.37 | 10.73 | 10.91 | 13.45 | 14.17 |
| 2 | 6.77 | 6.91 | 8.97 | 8.33 | 9.82 | 11.07 | 13.82 | 14.64 |
| 3 | 6.78 | 6.85 | 8.74 | 8.83 | 10.55 | 11.18 | 14.14 | 13.61 |
| 4 | 6.40 | 6.76 | 8.58 | 8.05 | 10.61 | 10.63 | 13.57 | 14.22 |
| 5 | 6.94 | 6.71 | 8.14 | 8.24 | 10.97 | 10.46 | 14.22 | 14.67 |
| 6 | 6.85 | 6.42 | 8.06 | 8.94 | 10.75 | 10.98 | 14.58 | 14.63 |
| 7 | 7.01 | 6.83 | 8.57 | 8.47 | 10.82 | 10.74 | 13.74 | 13.94 |
| 8 | 6.90 | 7.05 | 8.72 | 8.69 | 10.57 | 10.67 | 14.05 | 14.15 |
| 9 | 6.78 | 6.84 | 8.63 | 8.73 | 10.33 | 10.63 | 14.26 | 14.11 |
| 10 | 6.81 | 6.90 | 8.76 | 8.65 | 10.63 | 10.75 | 14.16 | 14.06 |
| Average | 6.85 | 6.83 | 8.51 | 8.53 | 10.58 | 10.80 | 14.00 | 14.22 |

| | | | | |
|---|---|---|---|---|
| UF/ED | 100.3% | 99.76% | 97.96% | 98.45% |

Table 5 shows that the measurement results of the FT4 obtained by ultrafiltration and equilibrium dialysis are consistent at a plurality of different temperature conditions. In other words, the ultrafiltration and the equilibrium dialysis have consistent results of FT4 at the same temperature. Therefore, the equilibrium dialysis at 37° C. can be used as reference to establish a conversion formula between the ultrafiltration at different temperatures (for example, 20° C., 25° C., and 30° C.) and the equilibrium dialysis at 37° C. Finally, the present patent achieves the content conversion of FT4 by transforming ultrafiltration at different temperatures (for example, 20° C., 25° C., and 30° C.) to the concentrations obtained by equilibrium dialysis method at 37° C. or ultrafiltration method at 37° C.

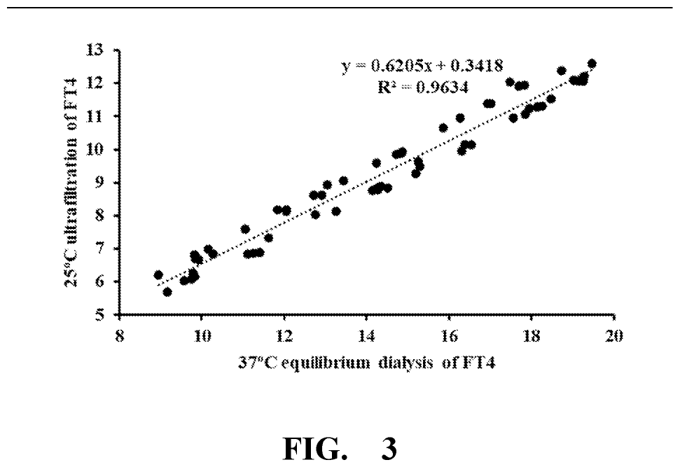

For ease of further understanding, in this embodiment, comparison the concentrations of FT4 between ultrafiltration at 25° C. and equilibrium dialysis at 37° C. is merely used as an example (similar comparison may be performed at other temperatures). In detail, concentrations of FT4 in 60 samples are measured by ultrafiltration at 25° C. and equilibrium dialysis at 37° C., respectively. Measurement results are shown in Table 6, and an obtained calculation formula is shown in FIG. 3. Table 6 Measurement results of FT4 in 60 different serum specimens under 25° C. ultrafiltration (UF)

TABLE 6
Measurement results of FT4 in 60 different serum specimens under 25°
C. ultrafiltration (UF) and 37° C. equilibrium dialysis (ED)

| No. | 1st conc. (UF) | 2nd conc. (ED) |
|---|---|---|
| 1 | 8.78 | 14.28 |
| 2 | 11.54 | 18.47 |
| 3 | 6.04 | 9.58 |
| 4 | 6.22 | 8.95 |
| 5 | 6.84 | 10.28 |
| 6 | 11.94 | 17.84 |
| 7 | 8.85 | 14.29 |
| 8 | 8.03 | 12.75 |
| 9 | 9.85 | 14.72 |
| 10 | 12.07 | 19.25 |
| 11 | 10.15 | 16.39 |
| 12 | 6.69 | 9.84 |
| 13 | 6.15 | 9.82 |
| 14 | 5.69 | 9.16 |
| 15 | 11.32 | 18.27 |
| 16 | 12.08 | 19.02 |
| 17 | 8.13 | 13.26 |
| 18 | 6.87 | 11.26 |
| 19 | 12.21 | 19.28 |
| 20 | 12.38 | 18.73 |
| 21 | 8.76 | 14.15 |
| 22 | 6.67 | 9.93 |
| 23 | 7.33 | 11.62 |
| 24 | 9.26 | 15.20 |
| 25 | 8.62 | 12.92 |
| 26 | 7.59 | 11.05 |
| 27 | 10.94 | 17.56 |
| 28 | 6.25 | 9.79 |
| 29 | 6.99 | 10.15 |
| 30 | 11.38 | 16.93 |
| 31 | 12.04 | 17.48 |
| 32 | 8.83 | 14.52 |
| 33 | 6.84 | 11.12 |
| 34 | 12.07 | 19.16 |
| 35 | 8.18 | 11.84 |
| 36 | 9.05 | 13.43 |
| 37 | 8.61 | 12.72 |
| 38 | 12.60 | 19.48 |
| 39 | 8.92 | 13.05 |
| 40 | 8.83 | 14.25 |
| 41 | 9.93 | 14.86 |
| 42 | 9.48 | 15.29 |
| 43 | 9.59 | 14.23 |
| 44 | 10.15 | 16.54 |
| 45 | 8.12 | 12.05 |
| 46 | 8.89 | 14.36 |
| 47 | 10.95 | 16.26 |
| 48 | 11.28 | 18.15 |
| 49 | 11.06 | 17.85 |
| 50 | 11.93 | 17.69 |
| 51 | 6.81 | 9.82 |
| 52 | 6.10 | 9.75 |
| 53 | 10.65 | 15.86 |
| 54 | 6.88 | 11.40 |
| 55 | 11.24 | 17.94 |
| 56 | 9.64 | 15.26 |
| 57 | 9.89 | 14.82 |
| 58 | 11.39 | 17.01 |
| 59 | 9.94 | 16.31 |
| 60 | 8.18 | 12.05 |

The results indicate that the concentrations of FT4 in 60 serum specimens are acquired by ultrafiltration at 25° C. for 1 h and equilibrium dialysis at 37° C. for 22 h, respectively, to obtain ultrafiltrate (first concentrations, 1st conc.) and equilibrium dialyzate (second concentrations, 2nd conc.), and finally, a related linear equation is obtained. The linear equation of the FT4 is finally calculated based on the results of 25° C. ultrafiltration and the results of 37° C. equilibrium dialysis, which is y=0.6205x+0.3418, where R²=0.9634, and n=60. The coefficient of determination R² is greater than 0.95, which indicates good correlation between the results of 25° C. ultrafiltration and the results of 37° C. equilibrium dialysis. Then, the concentration of FT4 at 37° C. (ultrafiltration or equilibrium dialysis) may be calculated based on the result of 25° C. ultrafiltration, and calculation formulas are as follows:

$$the first concentration ontained at 25 ° C . ultrafiltration = the second concentration obtained at 37 ° C .$$

$$equilibrium dialysis \times 0.6205 + 0.3418 ; and$$

$$a sample concentration = ( the first concentration - 0.3418 ) / 0.6205 .$$

The foregoing embodiments represent only preferred implementations of the present invention, and the descriptions thereof are relatively specific and detailed, which shall not be construed as limitations on the scope of the present invention. Furthermore, it should be noted that ordinary technicians in the same/similar fields can further develop some deformations, improvements, and/or substitutions without deviating from the concept of the present invention, of which, these changes are all within the protection scope of the present invention. Therefore, the protection scope of the invention patent shall be subject to the appended claims.

What is claimed is:

1. A content conversion method of free components by transforming ultrafiltration to equilibrium dialysis, comprising the following steps:
a step of separating the free components from samples by ultrafiltration and measuring concentrations of free components, to obtain first concentration;
a step of separating the free components from samples by equilibrium dialysis and measuring concentrations of free components, to obtain second concentration; and
a step of establishing a linear equation based on the first concentration and the second concentration.

2. The content conversion method of free components by transforming ultrafiltration to equilibrium dialysis according to claim 1, wherein there are a plurality of samples, and the quantity of samples is sufficient for a correlation coefficient of the established linear equation to be greater than 0.90.

3. The content conversion method of free components by transforming ultrafiltration to equilibrium dialysis according to claim 1, wherein high-performance liquid chromatography tandem mass spectrometry is used for the step of measuring concentrations.

4. The content conversion method of free components by transforming ultrafiltration to equilibrium dialysis according to claim 1, wherein the first concentration and the second concentration are measured at different temperatures.

5. The content conversion method of free components by transforming ultrafiltration to equilibrium dialysis according to claim 4, wherein the first concentration is measured at 4° C. to 37° C.; and the second concentration is measured at 4° C. to 37° C.

6. The content conversion method of free components by transforming ultrafiltration to equilibrium dialysis according to claim 1, wherein the specimen is blood, saliva, or urine.

7. The content conversion method of free components by transforming ultrafiltration to equilibrium dialysis according to claim 6, wherein the specimen is serum or plasma.

8. The content conversion method of free components by transforming ultrafiltration to equilibrium dialysis according to 1, wherein the free components are free hormones.

9. The content conversion method of free components by transforming ultrafiltration to equilibrium dialysis according to 8, wherein the free hormones are free testosterone, free triiodothyronine, or free thyroxine.

10. The content conversion method of free components by transforming ultrafiltration to equilibrium dialysis according to 9, wherein when the first concentration is measured at 25° C. and the second concentration is measured at 37° C.,
the linear equation is y=0.8719x−0.2116 when the free hormone is free testosterone;
the linear equation is y=0.7745x+0.066 when the free hormone is free triiodothyronine;
the linear equation is y=0.6205x+0.3418 when the free hormone is free thyroxine; and
y represents the first concentration, and x represents the second concentration.
